(19) United States
(12) Patent Application Publication
(10) Pub. No.: US 2018/0071407 A9
(43) Pub. Date: Mar. 15, 2018

(54) SYSTEMS AND METHODS FOR INTRA-TARGET MICRODOSING (ITM)

(71) Applicant: Tal Burt, Durham, NC (US)

(72) Inventor: Tal Burt, Durham, NC (US)

(21) Appl. No.: 14/488,655

(22) Filed: Sep. 17, 2014

Related U.S. Application Data
(60) Provisional application No. 61/880,083, filed on Sep. 19, 2013.

(51) Int. Cl. A61K 49/00; A61B 5/00; A61M 5/172; A61B 5/02; A61B 5/20; A61B 5/145; A61B 5/04; G01N 33/50; A61K 51/04

(52) U.S. Cl. CPC A61K 49/0004; A61B 5/4848; A61M 5/1723; A61B 5/02007; A61B 5/4244; A61B 5/201; A61K 51/0491; A61B 5/425; A61B 5/02028; A61B 5/14546; A61B 5/14532; A61B 5/04001; G01N 33/5088; A61B 5/4064

(57) ABSTRACT

Systems and methods for intra-target microdosing are disclosed. According to an aspect, a method for intra-target microdosing includes administering a microdose of drug into target tissue within a subject. The method also includes measuring efficacy of the administered drug to the target tissue.

### CROSS REFERENCE TO RELATED APPLICATION

This application claims priority to and the benefit of U.S. Provisional Patent Application No. 61/880,083, filed Sep. 19, 2013 and titled SYSTEMS AND METHODS FOR INTER-ARTERIAL MICRODOSING, the disclosure of which is incorporated herein by reference in its entirety.

### TECHNICAL FIELD

The present subject matter relates to medicine. More particularly, the present subject matter relates to intra-target microdosing (ITM).

### BACKGROUND

Microdosing is an approach to early drug development where exploratory pharmacokinetic data are acquired in humans using inherently safe sub-pharmacologic doses of drug. The technique of microdosing may be used to assess human pharmacokinetics prior to full Phase I clinical trials. Whilst other techniques of pharmacokinetic prediction rely on extrapolation of data from in silico, in vitro, or in vivo preclinical models, microdosing obtains data directly from the target species—that is human. Based upon the adage that “human is the best model for human” microdosing offers an inherently safe way of obtaining exploratory pharmacokinetic data from humans, primarily to enable the elimination of drugs from entering costly full development programs at the earliest possible stage in the clinical studies.

Advantages of microdosing include, for example, that an inherently safe microdose can be administered based upon a reduced preclinical safety package, costing approximately 10% of that required to enter Phase I allowing human study to be completed in 4-6 months and prior to traditional IND. Further, microdosing can allow the safe study of drugs in vulnerable populations (e.g., children, pregnant women, hepatically-/renally-impaired, and elderly) who are routinely excluded from clinical trials due to safety concerns. Microdosing can offer better prediction of human PK than alternative pre-clinical methods. As indicated in our published analysis of microdosing data (Lappin, Noveck, and Burt, 2013), 79% of orally administered and 100% of IV administered microdoses were scalable to full-dose PK. Traditional preclinical development has been estimated to predict human PK in only 45% of cases. Further, for example, microdosing requires only a fraction of the drug amount to be manufactured (grams versus kilograms required for full-dose clinical trials) in non-GMP conditions. Microdosing can always be administered IV. Microdosing also allows testing of drug-drug interactions and pharmacogenomic divergences among individuals without exposure to toxic drug effects (i.e., personalized medicine approach). Microdosing can enable academic drug developers to test their drugs in humans—usually a prohibitive undertaking due to financial constraints and regulatory complexities. Further, by enabling elimination of drugs before animal testing, microdosing can reduce the unnecessary use of animals for human drug development.

In view of the foregoing, it is desired to provide advancements and improvements to microdosing systems and techniques.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In accordance with embodiments of the present disclosure, intra-target microdosing is disclosed herein as a novel drug development approach. Drug development is risky, lengthy, expensive, and error-prone process. Amongst approaches recently proposed to address these challenges is microdosing, the testing in humans of sub-pharmacological doses prior to traditional Phase-1 to gain initial insights into drug response in a safe manner. The techniques disclosed herein provide quicker and lower cost human testing, informed selection amongst multiple pre-clinical analogues, and ability to study old drugs in vulnerable populations (e.g., children and elderly). The main challenges facing microdosing are (1) only pharmacokinetic (PK) data can be obtained and (2) uncertainty about extrapolation of microdose data to full-dose range. IAM combines features of microdosing with intra-target delivery to address these challenges. By administering microdose, calculated on a total-body basis, into an artery supplying a small region, full-dose exposure is generated in that region before returning systemically as microdose. Thus, two studies are combined: (A) IAM study whereby target is exposed to pharmacological concentrations and (B) “traditional” microdosing study through the dose returning systemically. Target pharmacological and systemic microdose exposures are compared in the same individuals at the same time thus reducing variability. IAM infusion profile could be modified by changing the following parameters: test article concentration, infusion duration, amount of fluid infused, type of fluid infused, and temporary reduction of venous outflow. IAM provides crucial pharmacodynamic (PD) data relevant to drug safety and efficacy. IAM also helps validate PK extrapolation from microdose to full-dose. Target exposure data obtained using IAM together with PBPK modeling can help determine linear relationship between microdose and full-dose, and, in case the relationship is not linear, help identify the type of non-linearity and mechanisms responsible for it. In either case predictability of the full-dose can be enhanced. This can inform Phase-1 study dose selection and can increase the value of these studies.

Disclosed herein are systems and methods for inter-arterial microdosing. According to an aspect, a method for intra-target microdosing includes administering a microdose of drug into target tissue within a subject. The method also includes measuring efficacy of the administered drug to the target tissue.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a diagram of an example intra-target microdosing (ITM) system in accordance with embodiments of the present disclosure;

FIG. 2 is a diagram showing a comparison of a conventional drug development process with intra-target microdosing in accordance with embodiments of the present disclosure;

FIG. 3 is a diagram of an example system and method of insulin IAM infusion at the radio artery in accordance with embodiments of the present subject matter;

FIG. 4 is a graph showing a hypothesized ¹⁸F-FDG hand uptake with and without insulin IAM; and

FIG. 5 is a flow diagram showing example IAM development progression.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

Disclosed herein are systems and methods for inter-arterial microdosing. The present disclosure can be utilized to test new drugs in target organs and tissues of interest. By use of the systems and methods disclosed herein, new drugs can be developed with less risk in humans, including vulnerable populations (such as children, pregnant women, elderly frail, hepatically-impaired, and renally-impaired), and brought to those who need them earlier than is possible with traditional approaches. An advantageous feature of the present subject matter is that when a microdose (defined as less than 1/100^{th} of the total body minimal pharmacological dose or less than 100 μg) is delivered via an artery into a body area 1/100^{th} of the total body mass or less, a temporary pharmacological concentration can be generated in the tissue or organ of interest. This may be sufficient to detect a biomarker and collect pharmacodynamic (PD) data relevant to the drug's safety and efficacy in addition to the pharmacokinetic (PK) data that traditional microdose studies provide. The drug may subsequently enter the systemic circulation via local venous drainage as a systemic microdose. Use of systems and methods as disclosed herein can add PD data and the validity of full pharmacological dose measurements to the existing advantages of microdosing.

IAM combines the methodologies and techniques of ‘microdosing’ and ‘intra-target drug delivery’ to test new drugs in target organs and tissues of interest. It produces a full pharmacological exposure in the organ/tissue of interest and a microdose in the rest of the body. Since a microdose is defined as 1/100th of the anticipated pharmacological dose calculated on a total-body weight basis, when such a dose is administered into an artery supplying 1/100th of the body mass or less, pharmacological concentrations are briefly (up to an hour) generated in the target tissue before entering the general circulation as a sub-pharmacological dose (microdose). The plan can be to test the organ/tissue of interest at pharmacological-level exposure corresponding to the (eventual) effective oral (or IV) doses. The doses given intra-target can start very low (sub-pharmacologically) and be titrated gradually until the first PD indications in the organ/tissue of interest. This can ensure no excessive exposure in the tissue of interest. Drug could be administered at constant or varied infusion rates with different durations and concentrations to reflect desired tissue exposure-response profiles and modelling requirements. The temporary pharmacological concentration generated in the tissue or organ of interest may be sufficient to detect changes in local biomarkers and collect PD data relevant to the drug's safety and efficacy. In addition, PK data, in the tissue of interest (via PET-imaging) or systemically (via venous sampling independent from the one associated with the IAM sampling), can be obtained as in conventional microdose studies, thereby maximizing the body of informative data from a single clinical study. Since the dose is administered by a vascular route, estimates of disposition kinetics, such as volume of distribution (V) and clearance (CL) could be obtained. These parameters can be important for drug developers, particularly where there is uncertainty in the prediction of clearance from non-human based methods such as allometry. Moreover, data to date suggests that prediction of V and CL for microdose studies is highly reliable. To our knowledge this is the first description of IAM and the first description of intra-target drug delivery in drug development but related approaches that can be described as ‘targeted investigational medicine’ have been reported, albeit without measurement of test article concentrations in the tissues or vessels of interest.

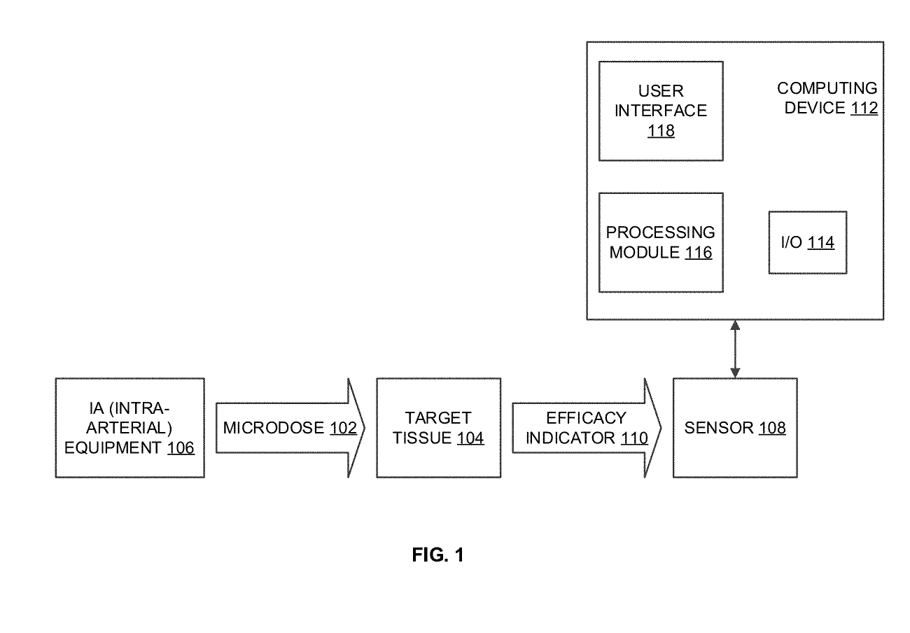

FIG. 1 illustrates a diagram of an example intra-target microdosing (ITM) system 100 in accordance with embodiments of the present disclosure. Referring to FIG. 1, the target may be within a subject, such as a human. A drug microdose 104 may be administered or otherwise introduced into target tissue 104 of the subject. For example, the target may be an artery of the subject. In other examples, the target may be a target organ or other target tissue of the subject. Example targets include, but are not limited to, peripheral vascular, periphery, liver, kidney, heart, brain, pancreas, blood, and central nervous system (CNS). The target may be, for example, a body area of about 1/100^{th} of the total body mass of the subject. In an example, the microdose of drug may be about 1/100^{th} or less of a total body minimal pharmacological dose for the subject. In another example, the microdose of drug may be about 100 μg or less. The microdose of drug may be administered by any suitable intra-target (IT) equipment 106 or other suitable drug-administering equipment. For example, the microdose may be administered into targeted tissues by techniques other than intra-target administration such as, but not limited to, intra-muscular, intra-thecal, intra-bronchial, intra-nasal, subcutaneous, intra-dermal, intra-occular, and by injection directly into the parenchyma of an organ (e.g., intra-hepatic).

Table 1 below shows examples of intra-target microdosing applications.

TABLE 1
Examples of IAM Applications

| Drug | Organ/Tissue | Biomarker |
|---|---|---|
| Nitrates, Inotropes, | Peripheral Vascular | Vasodilation, |
| Adrenergic, Muscarinic, PDE5 | | Vasoconstriction, cGMP |
| inhibitors, neutral | | spillover measurement |
| endopeptidase (NEP) | | |
| inhibitors, natriuretic peptides | | |
| Analgesics and Anesthetics | Peripheral Organ/Tissue | Analgesia and Anesthesia |
| Triptans | Blood vessels | Analgesia, substance P and |
| | | CGRP levels |
| Chemotherapy | Liver, Kidney, Brain, | Receptor Binding (with PET |
| | Pancreas, Breast | imaging of radiolabeled drug) |
| Neuromuscular Blocking | Skeletal Muscles | Muscle Relaxation/Paralysis |
| Agents | | |
| Anticoagulants and | Blood | Coagulation Parameters, |
| Antiplatelet | | Platelet Aggregation |
| Immune modulators and | Blood | Cytokines, Allergic Symptoms |
| Antihistamines | | |
| Hypoglycemics, Sodium | Kidney | Glucose levels, Reabsorption |
| Glucose | | in proximal tubule (by ¹⁸F- |
| Cotransporter-2 (SGLT-2) | | FDG) |
| Inhibitors, Diuretics | | |
| CNS Stimulants and | CNS | Neuronal Activity (e.g., Wada |
| Depressants (e.g., Hypnotics, | | Test) |
| sedatives, anxiolytics), NMDA | | |
| Antagonists | | |

Subsequent to administering the microdose, the efficacy of the administered drug to the target tissue may be measured. Referring to FIG. 1, a sensor 108 may be suitably configured with a subject for measuring the efficacy of the administered microdose. The measure may be referred to as an efficacy indicator 110. As an example, a biomarker (also referred to as a “biological marker”) may be suitably measured. Example biomarkers include, but are not limited to, vasodilation, vasoconstriction, analgesia, receptor binding, coagulation parameters, platelet aggregation, cytokines, glucose absorption in proximal tubule, and neuronal activity. Any suitable surrogate biomarkers may be utilized such as, but not limited to, intermediate biomarkers linking target/tissue drug exposure with the ultimate therapeutic outcomes, could also serve as biomarkers for the IAM approach. The biomarker may be a traceable substance introduced into the subject as a way to examine organ function or other aspects of health. The biomarker measured by the sensor 110 may be indicative of the effectiveness of the microdose 102 introduced to the target tissue 104. It is also noted that microdose effectiveness may be determined by measurement of more than one biomarker.

Biomarkers may be captured in a short time frame on the order of seconds to minutes. Further, biomarkers may be predictive of information obtained when full pharmacological dose is administered systemically. Table 1 above lists several example applications of IAM in addition to hormonal system application (insulin). These applications can generate biomarkers relevant to drug action within second or minutes. In addition, demonstration of target receptor binding through PET-imaging can occur rapidly, is relevant to many types of drug candidates, and represents powerful mechanistic support of drug actions relevant to safety and efficacy. It is noted that these examples should not be considered limiting, as it should be understood to those of skill in the art that the systems and methods disclosed herein may be suitably used in other applications.

By delivery of a microdose via an artery in a body area 1/100^{th} of the total body mass, a temporary pharmacological concentration can be generated in the tissue or organ of interest. This may be sufficient to detect a biomarker and collect pharmacodynamics (PD) data relevant to the drug's safety and efficacy in addition to pharmacokinetic (PK) data. In this way, efficacy of the administered drug to the target tissue may be measured. The drug may subsequently enter the systemic circulation via local venous drainage as a systemic microdose.

With continuing reference to FIG. 1, the system 100 may include a computing device 112 that is operatively connected to the sensor 108. The computing device 112 may be in communication with the sensor 108. The sensor 108 may generate and output to the computing device 112 an electrical signal representative of the efficacy indicator 110. The computing device 112 may include an input/output module 114 configured to receive the electrical signal and to convert the received signal into data suitable for processing. A processing module 116 of the computing device 112 may be configured to process the data, to analyze the data, and to present analysis results to a user. For example, the processing module 116 may control a user interface 118 to present text and/or graphics to a user via a user interface 118.

As referred to herein, the term “computing device” should be broadly construed. A computing device can be a desktop computer, a laptop computer, or a tablet computer. In other example, a computing device can include any type of mobile computing device, for example, a smartphone, a cell phone, a pager, a personal digital assistant (PDA, e.g., with GPRS NIC), a mobile computer with a smartphone client, or the like. These devices include user interfaces for interaction with a user.

As referred to herein, a “user interface” is generally a system by which users interact with a computing device. A user interface can include an input for allowing users to manipulate a computing device, and can include an output for allowing the system to present information and/or data, indicate the effects of the user's manipulation, etc. An example of an interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen at which the display icon is displayed for selecting the display icon. In another example, the user can use any other suitable interface of a computing device, such as a keypad, to select the display icon or display object. For example, the user can use a track ball or arrow keys for moving a cursor to highlight and select the display object.

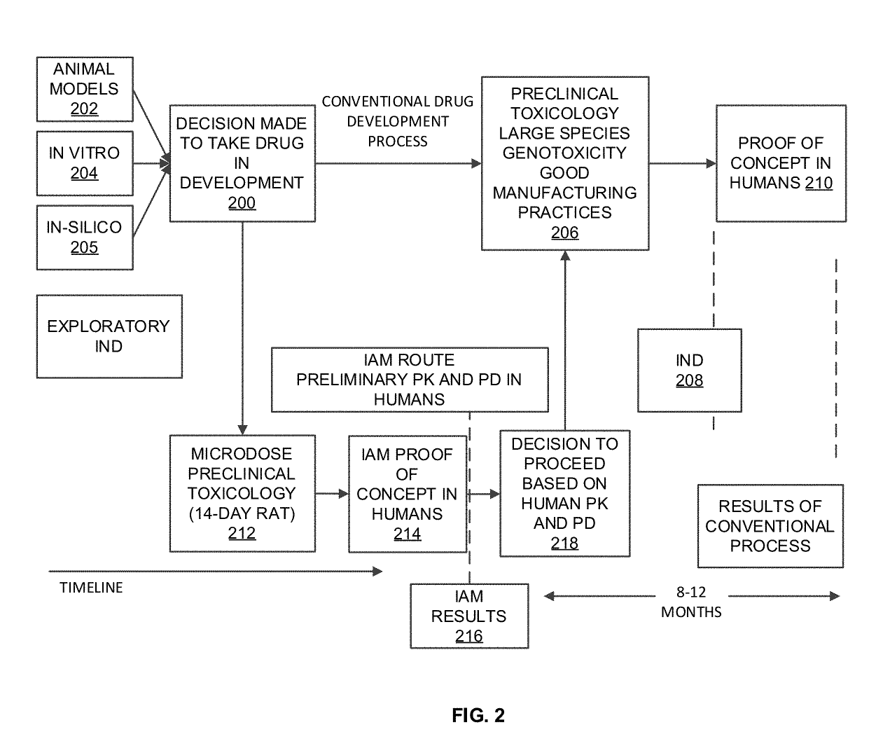

FIG. 2 illustrates a diagram showing a comparison of a conventional drug development process with intra-target microdosing in accordance with embodiments of the present disclosure. Referring to FIG. 2, a conventional drug development process typically involves making a decision 200 to take a drug into development by using animal models 202, testing in vitro 204, and/or testing in-silico 205. Subsequently, the conventional process includes testing in living animals (usually rodents and dogs) 206. As a result, an investigational new drug (IND) can be developed 208. Afterwards, the conventional process may involve suitable techniques for performing proof of concept in humans 210. Only one species (usually rodent) is required for the IAM approach. In the conventional approach genotoxicology studies on the effects of the drug on genetic material in the living animal are also necessary but not required for the IAM approach. The traditional approach calls for large quantities (kg amounts) of the new drug which may require establishment of a production process and facilities that would not be necessary for the IAM approach.

In an IAM process in accordance with the embodiments of the present disclosure, subsequent to a decision being made to develop a drug at step 200, the IAM process may include administering microdoses in a preclinical setting to animals (e.g., 14-day rats or other rodents) 212. After obtaining satisfactory results in step 212, IAM may be administered to humans 214 for proof of concept. IAM results may be obtained 216 and a decision made 218 based on the IAM results. IAM can enable a collection of biomarkers PD data relevant to drug safety and efficacy in addition to tissue and systemic PK data. Pharmacological dose data generated with IAM in organs and tissues of interest can enhance the validity and reliability of extrapolation, and predictability of the approach. With IAM, new drugs can be tested in humans (including vulnerable populations) in a safe and cost-effective manner 8-12 months earlier than conventional IND approaches.

IAM in accordance with embodiments of the present subject matter can generate various data relevant to pharmacological drug response. For example, IAM can be used to generate pharmacodynamic data in target organs/tissues. This can be determined by measurement of biomarkers in plasma samples from venous drainage of the target organ/tissue. In another example, IAM can be used to generate pharmacokinetic data in target organs/tissues and systemic circulation. This can be determined by measurement of drug concentrations in plasma samples from venous drainage of the target organ/tissue and the systemic circulation. Further, for example, IAM can be used to generate positron emission tomography (PET) imaging data of target organs/tissues and whole body, and to generate respective PK and PD data based on standard uptake value (SUV) measurements converting radioactive signals to tissue concentrations as set forth in the following formulae:

$$\begin{matrix}SUV = \frac{Local Radioactivity Concentration}{( \frac{Administered Radioactivity}{Body Weight} )} & Formula 1 \\ Local Drug Concentration = SUV ( \frac{Administered Drug}{Body Weight} ) & Formula 2\end{matrix}$$

The safety of the IAM approach may be maintained through the brief (e.g., seconds to minutes) exposure through infusion of the drug into a local artery, and the limited portion ( 1/100th the body mass) that is exposed to the full dose. Safety may also be ensured by the ability to interrupt the infusion immediately upon indication of any toxic effects. In an example, Macaque monkeys may be chosen for the first step due to the novelty of the approach to establish safety and technical feasibility and test methodological assumptions. Further, for example, the hand may be chosen as a model of peripheral drug action, being less than 1/100 of the body mass (about 0.6%), familiarity with radial artery cannulations, mobility and minimal invasiveness. Further, for example, regular insulin may be chosen for known physiological and disease model, known and quick-response biomarkers, abundance of reference information, relevance to the hand, ease of IV and IA administration and known antidote. Insulin has been previously administered intra-arterially in the forearm. ¹⁸F-FDG may be chosen due to quick visualization of glucose uptake—an effective biomarker of insulin response. Sample and imaging time points may include initial time points (0, 5, 10, 20, 30 minutes) for sample collection and PET imaging can be used based on the known half-life of insulin (4-6 minutes). Observation duration totals 5 times insulin half-life, an accepted standard for the measurements of drug pharmacokinetics. Adjustments to these time-points can be made after initial inspection of the data with aim to provide optimal characterization of PK and PD profiles. Insulin may be infused over the duration of the sample collection and imaging time points. The purpose of this is to simulate the continuous perfusion pattern that follows systemic administration. Regarding sample size, no power calculation may be used to determine the sample size. The choice of human and primate sample size (e.g., 6) may be done to reflect typical sample sizes in microdosing studies. Regarding on-randomization and absence of control group, due to the small sample size and pilot nature of the study, respectively. Nevertheless, in the PET-imaging component the contralateral hand can function as control of the ipsilateral hand in the same individual.

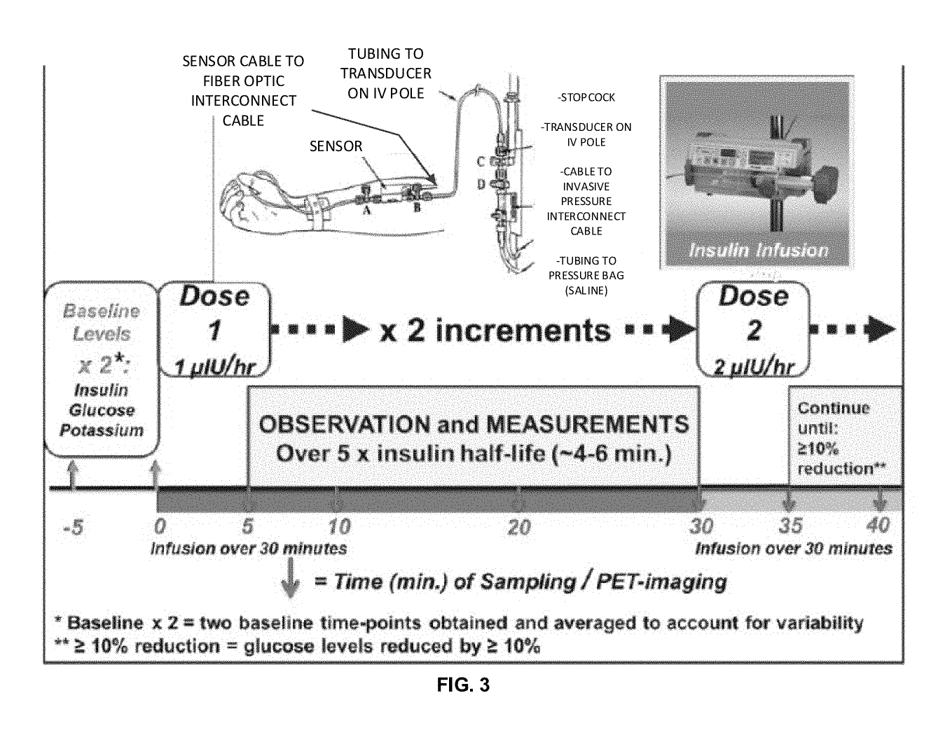

FIG. 3 illustrates a diagram of an example system and method of insulin IAM infusion at the radio artery in accordance with embodiments of the present subject matter. This example may be implemented by the system 100 shown in FIG. 1 or any other suitable system. Referring to FIG. 3, the extent to which peripheral IAM reflects plasma PK & PD post systemic pharmacological dose administration may be determined. The effects of systemic (IV) administration of pharmacological concentrations of insulin may be compared to the effects produced by the intra-target delivery of microdose. In each case, arterial and venous concentrations of insulin, glucose, and potassium may be measured before and after insulin infusions at time points 0, 5, 10, 20, and 30 minutes (see FIG. 3). It may be expected that IAM can cause reductions in localized plasma glucose and potassium concentrations from veins draining the hand, similar to reductions produced after systemic administration of pharmacological doses. Likewise, it may be expected that insulin plasma concentrations after IAM administration may be similar to those after systemic administration. Initial testing may be in non-human primates, which is followed by human volunteers. Insulin infusions can start in sub-pharmacological range and titrate up until changes in the biomarkers are detected. Intra-arterial microdose infusions (see FIG. 3) may be followed by systemic pharmacological dose infusions.

The starting IAM infusion rate may be calculated on the basis of the lowest systemic pharmacological insulin infusion rate (1 IU/hour) divided by 100 to obtain the microdose. Since the hand is about 1/100^{th} of the body mass, this microdose infusion level is the approximate expected exposure of the hand when a systemic insulin infusion is given at 1 IU/hour. This can be divided by additional factor of 10 (as a safe, conservative estimate of the minimal effective dose in the hand) to establish the starting IAM infusion rate: 1 mIU/hour (see FIG. 3). If the first infusion produces biomarker changes, lower doses (at 2-fold reductions each) will be administered until minimal dose (i.e., no response) is detected.

Reduction in glucose levels may be the primary biomarker for this study and may be defined as 10% or greater decrease in venous glucose concentrations.

Glucose and potassium levels can be measured at bedside for titration decision-making (using Radiometer ABL90, measurement errors are 5.6% and 6.4%, respectively). Bedside measurements can be verified with values obtained through a suitable PK/PD bioanalytical core lab. Insulin in human, macaque, and rat serum can be measured by commercially available ELISA kits (e.g., Millipore Inc, Biotang Inc., and Diazyme Inc., respectively). Potassium and glucose in serum (in all three species) will be measured by commercially available enzymatic colorimetric assay kit (CRYSTAL CLEAR Inc) and ELISA kit (Cayman Chemical Inc.). Human blood can be collected into Vacutainer tube (BD #366668, red cap, 3 mL) and blood can be allowed to clot at RT for 30 minutes followed by centrifugation at 1500×g for 15 minutes. The clear serum can be split into two ˜1 mL aliquots dispensed into labeled 2-mL polypropylene cryo-tubes and stored at −80° C.

During the systemic infusions of insulin, serial arterial blood samples can be obtained from the radial artery to measure plasma concentrations in order to determine the insulin concentrations that the hand is exposed to after systemic administration. These concentrations can be correlated with the concentrations being administered systemically. This can provide another estimate of dose-to-microdose proportionality. To ensure adequate comparisons of systemic and IAM administrations, IAM infusion concentrations can be normalized to the values obtained post systemic dose administration.

The lowest dose level at which glucose concentrations are reduced by ≧10% may be determined for IAM administration (IAM₁₀) and systemic administration (Sys₁₀). The ratio Sys₁₀/IAM may the primary outcome for in accordance with embodiments of the present disclosure.

In one experimental design, the non-human primate may follow the approach described above. Animals may be suitably prepped. Participants may undergo an informed consent process followed by screening procedures. Qualified individuals may be confined within a suitable clinical research unit from the day prior to the study until completion of the systemic and intra-arterial insulin infusions, and the ¹⁸F-FDG-Insulin-intra-arterial microdose PET imaging component of the study. Participants may fast from midnight until completion of the above procedures. On the morning of the first study day, subjects may have 3 venous catheters placed (e.g., two in the contralateral arm, and one in the ipsilateral forearm [non-dominant hand]). The intra-arterial cannula may also be placed in the ipsilateral forearm, into the radial artery. This placement may be done under ultrasound guidance and the use of a local anesthetic. Allen's Test may be performed to confirm ulnar artery patency and safety of radial artery cannulation. Participants with abnormal Allen's Test may be excluded from the study. Venous drainage of the hand is predominantly on the dorsum of the hand so the sampling catheter may be placed in the cephalic vein. An infusion of 5% dextrose in water (D₅W) may be started at a rate of 50 mL/h in the proximal contralateral vein for the purpose of safety and to administer the systemic infusions of insulin. The distal forearm catheters may be used for the collection of biomarker and insulin samples.

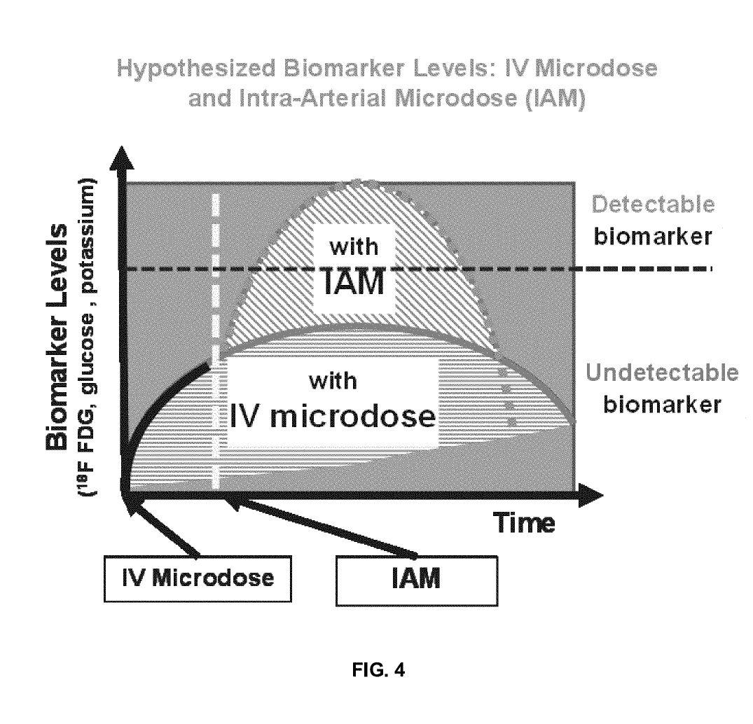

In another example, ¹⁸F-Fluorodeoxyglucose (FDG) uptake into both hands may be measured before and after IAM insulin infusion into the radial artery of the non-dominant hand. It may be expected that after IAM insulin infusion ipsilateral uptake of ¹⁸F-FDG is greater than baseline and contralateral uptake but comparable to uptake into both hands after systemic pharmacological dose insulin administration. For example, FIG. 4 illustrates a graph showing a hypothesized ¹⁸F-FDG hand uptake with and without insulin IAM. It can be expected that ¹⁸F-FDG uptake, measured with SUV (Formulae 1 and 2), will reflect both insulin PK and PD. To confirm, the results of ¹⁸F-FDG uptake may be compared to plasma insulin levels (for PK) and glucose uptake (for PD) in the veins draining the hands.

¹⁸F-FDG PET-imaging procedures may include: (1) administration of single IV dose of 5 mCi ¹⁸F-FDG; (2) 5 minutes for systemic equilibration; (3) 5-minutes scan of the ipsilateral hand followed by 5-minute scan of the contralateral, control hand; (4) insulin IV administration; (5) five-minute ipsilateral hand scan, followed by a 5-minute contralateral hand scan; (6) continue alternate scanning for at least 30 minutes (see FIG. 3); (7) obtain simultaneous IV blood sampling to match the scans; and (8) the procedures can be repeated on a separate study day post systemic administration of pharmacological insulin dose. Sequential post-baseline ipsilateral scans and ipsilateral venous samples may be compared with respective baseline and contralateral hand measurements. Non-human primates may be tested prior to human volunteers. The dose of insulin that produced biomarker changes in one study can be used in another study. Variability can be minimized by one study result following another. The procedure can be repeated on another day using the same systemic pharmacological insulin dose that produced biomarker changes in another study.

¹⁸F-FDG Uptake Change Definition—

Increase in uptake of ¹⁸F-FDG into the hands as measured by >25% or greater increase in tissue Standard Uptake Value (SUV^{19,31}).

Primary Outcome for Aim 2—

The amount of ¹⁸F-FDG uptake can be measured in SUV (Formulae 1 and 2) for both IAM and systemic dose administration. The ratio Systemic_{SUV}/IAM_{SUV} may be the primary outcome in accordance with embodiments.

In accordance with embodiments, a process may include determining the extent to which internal organ intra-arterial microdosing reflects plasma & tissue PK & PD post systemic pharmacological dose administration. Microdose insulin may be administered into the rat hepatic artery and measure insulin, glucose and potassium levels in the hepatic vein as in Aim 1 and PET imaging of ¹⁸F-FDG liver uptake. It can be expected that the microdose insulin infusion into the rat hepatic artery leads to observable changes in hepatic vein insulin, glucose, and potassium levels and liver uptake of ¹⁸F-FDG comparable with systemic pharmacological dose insulin administration.

¹⁸F-FDG can be administered systemically followed by insulin infusion into the hepatic artery. Serial sample collection and serial micro-PET images can initially follow procedures established in accordance with embodiments disclosed herein and adapt after initial inspection of the data to ensure optimal characterization of PK and PD parameters.

IAM in Rodent Liver:

Initially, the microdose experiment may involve one or two rats to determine the dose of insulin infused through the hepatic artery that is required to cause a decrease in glucose in the hepatic vein. The initial dose of 0.05 units of regular insulin may be infused over 5 minutes and 0.10 ml of blood may be collected from the hepatic vein for insulin, glucose and potassium measurements at 0 (immediately before infusion) and 5, 10, 20, 30 minutes after the infusion of insulin. Subsequent doses of insulin can be used, either increased or decreased in increments of 0.02 units, to determine the smallest dose of insulin that can cause a ≧10% decrease in blood glucose. When the smallest dose of insulin that may cause a ≧10% decrease in blood glucose is determined, 10 rats can undergo the procedure and 0.25 mls of blood can be collected from the hepatic vein, at the time points designated above, to determine insulin, glucose and potassium levels. To compare systemic response to the microdose one, a systemic dose of insulin can be set at hundred times the dose of insulin infused into the hepatic artery and images and blood samples obtained at 0, 5, 10, 20, 30 minutes after the infusion or at more appropriate time points as indicated by initial view of the data.

PET-Imaging of Rat Liver:

¹⁸F-FDG can be infused into the jugular vein cannula followed after 1 minute by insulin infusion over 1 minute into the hepatic artery. The liver can be imaged to determine glucose uptake.

Predictability can be assessed in terms of closeness of standardized treatment effects (e.g., reduction in glucose concentrations) at microdose and pharmacological dose in the target organ/tissue. Let T_{MD} and T_{PD} denote the response at microdose and pharmacological dose, respectively. It may be assumed that T_{MD} and T_{PD} follow distribution with means μ_{Tmd} and μ_{Tpd} and variances σ²_{Tmd} and σ²_{Tpd}, respectively. Then, the standardized treatment effects at microdose (ST_{MD}) and pharmacological dose (ST_{PD}) become:

$${ST}_{MD} = \frac{{T}_{MD} - {μ}_{Tmd}}{{σ}_{Tmd}} and {ST}_{PD} = \frac{{T}_{PD} - {μ}_{Tpd}}{{σ}_{Tpd}}$$

(where σ_{Tmd} and σ_{Tmd} are the standard deviations of responses at microdoses and pharmacological doses, respectively). The closeness can be tested by means of Power Analysis (not feasible in this preliminary investigation) or by means of Precision Analysis by obtaining 95% confidence interval (Δ_{low}, Δ_{high}) for sample mean for standardized microdose treatment effect (μ_{STmd}), and determining whether the sample mean of standardized treatment effect (μ_{STpd}) falls within (Δ_{low}, Δ_{high}).

Primary Outcome Measures:

insulin, glucose, potassium concentrations in a peripheral vein draining the hands and ¹⁸F-FDG uptake into the hand post insulin infusion. Input-rate (concentration-response relationship) can be determined. Demographics and patient characteristics can be summarized in terms of mean, standard deviation, median and range by dose level. At each time point (post-infusion), point estimates and the corresponding 95% confidence intervals for mean change from baseline (time 0) in insulin, glucose, and potassium levels and ¹⁸F uptake (SUV) can be obtained and compared for each dose level. A repeated measure analysis can be performed to compare the insulin, glucose, and potassium levels and ¹⁸F uptake (SUV) over time between IAM and systemic dose administration. In addition, ¹⁸F-FDG uptake can be compared between ipsilateral and contralateral hands. Incidence rate of adverse events and other relevant safety parameters can be examined and compared between IAM and systemic administration to provide a measure of the safety of the IAM approach. Intra-arterial measurements of insulin post systemic administration can be used to model the exposure of the hand to insulin and compare with the exposure post IAM.

Hypothesized Results: It can be anticipated that in the brief period of minutes after IAM, drug behavior in the target organ/tissue ‘just as’ or ‘sufficiently similar’ to that after systemic, full-dose, pharmacological dose administration may be observed. Specifically, it can be anticipated that the study may show that measurements of PK (insulin levels) and PD (biomarkers: glucose, potassium, and ¹⁸F-FDG uptake) after IAM may be reasonably (within 2-fold) similar to the same measurements after systemic insulin administration of pharmacological doses. This can prove the concept that IAM can be used to predict full, pharmacological dose behavior in humans. It can be anticipated that IAM may prove safe—with minimal adverse events due to intra-arterial cannulation. Together, these findings can support the feasibility and value of IAM in early drug development.

Various features of the present subject matter may be alternatively provided. To obtain optimal PK and PD profiles of insulin action and optimize signal detection from both plasma samples and PET-imaging, different infusion durations and spacing of time points may be provided. It is noted that PK and biomarker signals may be difficult to obtain in the rats due to the small doses given and limit on samples that can be drawn (10% of the animal blood volume). If so, a mini-pig may be utilized rather than a rat.

Confirmation of IAM safety, feasibility, and proof of concept can be followed by studies in other therapeutic areas, target organs and drug classes, both small molecules and proteins, and especially those belonging to BDDCS class 2 characteristic of modern drugs. Studies can be conducted with known drugs in healthy animals as well as in animal models of human disease. Following the stated lines of investigation, the present subject matter can first test in animals, in the periphery, then move to human in the periphery and animal internal organs and finally internal organs in humans and eventually in patients and vulnerable populations. Validation of findings may be obtained by use of internal organs of pigs. IAM testing in humans can continue in patients with existing intra-arterial lines for planned intra-arterial delivery of therapeutics or diagnostics who provide consent for testing of approved drugs in both microdose and therapeutic (pharmacological) doses. The data can be presented to regulators, demonstrating the safety and feasibility of IAM in predicting pharmacological dose response, to establish regulatory framework for the application of the approach in drug development.

Example benefits of human microdosing include, but are not limited to: short time from lab bench to completion of clinical studies (e.g., approximately 6 months); smarter lead candidate selection; reduces expensive late stage attrition—‘Kill Early—Kill Cheap’; substantially reduced preclinical toxicology package compared to Phase I; only gram quantities of non-GMP drug (e.g., about 10 g) are needed; any route of administration possible, including intravenous; absolute oral bioavailability calculation; drugs can be tested in sensitive populations, renally-impaired patients, women of child bearing age and cancer patients; DDI; personalized medicine—saving patients exposure to ineffective or unduly toxic drugs; and reduces use of animals in research.

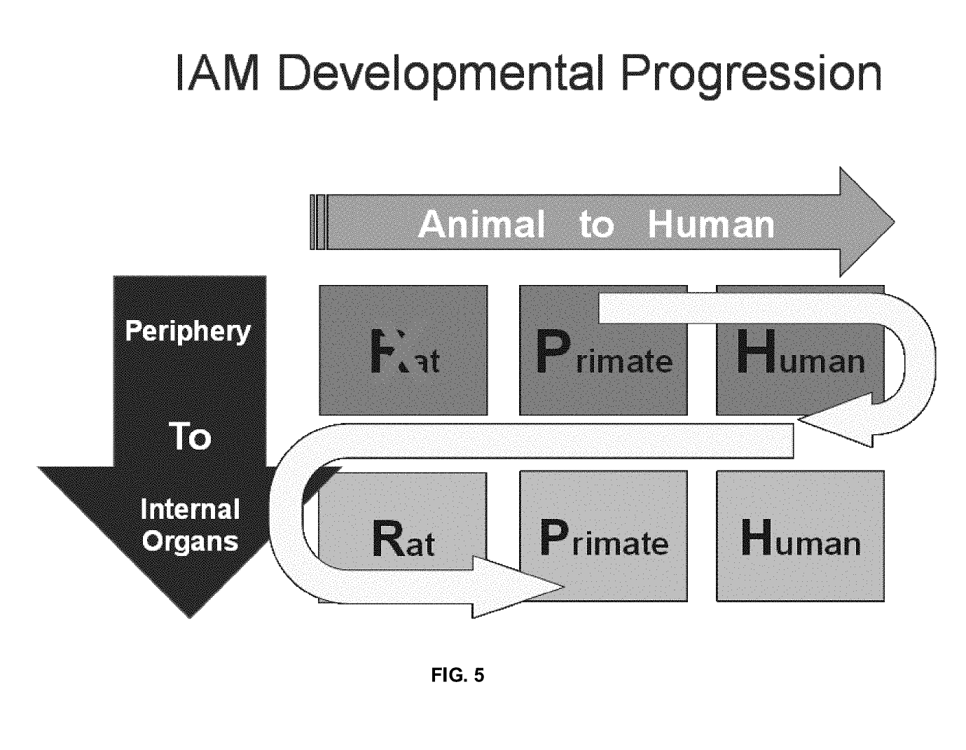

FIG. 5 illustrates a flow diagram showing example IAM development progression. Referring to FIG. 5, the serpentine arrow describes the steps in development: first animals in the periphery, humans in periphery (e.g., hand, skin), then internal organs liver) in animals, and then humans.

In accordance with embodiments of the present disclosure, various IAM approaches may be implemented. For example, a peripheral artery that is easily accessible and known to supply about 1/100th of the body mass is used. The peripheral tissue could be used to test systemic drug effects, i.e., those that are not organ or tissue-specific, such as the peripheral actions of thyroid hormones, immune modulators or vasodilators/constrictors. A suitable local artery could be the radial artery supplying the hand as the hand represents about 1/100th of the body mass. A vein draining the area may be cannulated for collection of plasma biomarkers.

In another example, an artery supplying an internal organ is cannulated together with the respective vein draining the organ. This may be preceded by angiography to determine arterial and venous distributions to ensure accessed area is within 1/100th of the body mass.

In another example, IAM could make use of existing arterial and or venous access during the course of diagnostic and/or therapeutic procedures (e.g., in the course of intra-arterial chemotherapy, or cardiac angiography). This would obviate the excess risk associated with the intra-arterial cannulation procedure.

In another example, access obtained in the course of surgery could facilitate study of organs or tissues that have challenging access such as tumors or small internal organs such as the pituitary.

In another example, biomarkers obtained when IAM is performed during surgery could include direct tissue visualization, physiological manipulation and testing, and histological examination of samples of healthy and/or pathological tissues exposed to the IAM intervention. Another example IAM application is tumor chemotherapy. In this example, a chemotherapeutic agent for hepatocellular carcinoma is administered into the hepatic artery. The drug or a biomarker (e.g., fluorodeoxyglucose [FDG]) is radiolabelled. Demonstration of binding to tumor tissue through PET imaging will be proof-of-concept that the chemotherapeutic agent reaches, penetrates and binds to cellular targets. Displacement studies could confirm binding to target of interest by administering competitive agonists. Serial PET-images obtained to show concentrations over time will be translatable to full-pharmacological exposure, intra-tumor PK, as well as systemic microdose PK. Chemical biomarkers could be obtained in the downstream vein. If done during surgery tissue samples of the tumor may be obtained for histological markers of therapeutic or toxic effects. Since the concentration returning to the systemic circulation becomes a microdose the traditional microdose study on systemic PK and binding to extra-hepatic tissues can be conducted as well.

In another example, IAM may be applied to SGLT-2 inhibitors and glucose excretion in the kidney. In this example, an SGLT-2 inhibitor (Sodium/Glucose Transporter Type 2 inhibitor) is administered via infusion into the renal artery. Glucose reabsorption into the proximal tubule is thus inhibited and glucose is excreted in the urine. The biomarkers would be the appearance of glucose in the urine, measured from samples obtained from the catheterized bladder, and by PET measurements of 18F-FDG reabsorption and excretion.

In another example, IAM may be applied to natriuretic peptides, vasodilation and cGMP as biomarkers. In this example, natriuretic peptide (analogue of the naturally occurring Atrial Natriuretic Peptide [ANP] and Brain Natriuretic Peptide [BNP]) may be administered into the radial artery. Hand vessel vasodilatation is measured using venous occlusion plethysmography and plasma cGMP concentrations are measured using enzyme immunoassay. Vasodilatation and cGMP concentrations serve as physical and chemical biomarkers, respectively, of drug action, and have been shown to take place within minutes of infusion.

In another example, IAM may be applied to neuromuscular blocking drugs (NMBD) and muscle strength. In this example, localized administration of an NMBD into the radial artery would have effects on hand muscles within seconds to minutes. Reduction in muscle strength (measured as percent ‘twitch response’, see below) is the biomarker associated with drug efficacy. Electrical stimulus is applied to the proximal ulnar nerve to induce a depolarization with the subsequent release of acetylcholine (Ach) at the neuromuscular junction, resulting in the contraction of the adductor pollicis muscle, termed the ‘twitch response’. This response is calibrated as a full twitch (100%). When the NMBD is administered, the reduction of the twitch can be graded (0-100%) where 0% represents full neuromuscular blockade. The pharmacodynamic action of the drug (onset, duration of blockade and offset) can then be recorded in terms of % twitch. This represents considerable advantage over the current NMBD development process whereby a dose is administered systemically with resultant complete muscle paralysis as NMBD have direct actions upon all striated muscle (including the laryngeal & accessory muscles of respiration) and smooth muscle (diaphragm). The subject would require full ventilatory support. In addition, NMBDs alone have no effect upon the level of consciousness, so the subject can be aware and likely experience the complete paralysis as an extreme anxiety-provoking event. In order to avoid the awareness problem, NMBD studies are conducted under general anesthesia. The utility of IAM would be the ability to administer NMBD via the radial artery to the target site of PD monitoring (adductor pollicis) without the concern of systemic concentrations capable of producing systemic paralysis and exposure to unnecessary and expensive additional procedures. Furthermore, reversal of NMBD residual muscular block is also of clinical importance and could also be studied in a safe and controlled manner using IAM in the early clinical development of NMBDs. This could prove of particular value in vulnerable populations such as children and elderly.

Although various that various example drugs and targets are described herein, these should not be considered limiting as any suitable drugs and targets may be utilized in accordance with embodiments of the present subject matter. As an example, any suitable target compound (e.g., insulin or another suitable drug) can be injected into the local artery and biomarkers (e.g., glucose levels or other biomarkers) may be measured in the vein draining the organ (e.g., hand, liver, or another target).

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the present subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present subject matter have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A method for intra-target microdosing, the method comprising:
administering a microdose of drug into target tissue within a subject; and
measuring efficacy of the administered drug to the target tissue.

2. The method of claim 1, wherein the target tissue includes one of an artery and an organ.

3. The method of claim 1, wherein the target tissue includes one of peripheral vascular, periphery, liver, kidney, brain, pancreas, blood, and central nervous system.

4. The method of claim 1, wherein the subject is a human.

5. The method of claim 1, wherein the target tissue is a body area about 1/100^{th} of the total body mass of the subject.

6. The method of claim 1, wherein the microdose of drug is about 1/100^{th} or less of a total body minimal pharmacological dose for the subject.

7. The method of claim 1, wherein the microdose of drug is about 100 μg or less.

8. The method of claim 1, wherein the drug is one of nitrates, inotropes, analgesics, chemotherapy, anticoagulants, immune modulators, sodium glucose, cotransporter-2 (SGLT-2), inhibitors, central nervous system stimulants, and depressants.

9. The method of claim 1, wherein measuring the efficacy comprises measuring a biomarker.

10. The method of claim 9, wherein the biomarker is one of vasodilation, vasoconstriction, analgesia, receptor binding, coagulation parameters, platelet aggregation, cytokines, glucose absorption in proximal tubule, and neuronal activity.

11. A system for intra-target microdosing, the method comprising:
drug-administering equipment configured to administer a microdose of drug into target tissue within a subject; and
a sensor configured to measure efficacy of the administered drug to the target tissue.

12. The system of claim 11, wherein the target tissue includes one of an artery and an organ.

13. The system of claim 11, wherein the target tissue includes one of peripheral vascular, periphery, liver, kidney, brain, pancreas, blood, and central nervous system.

14. The system of claim 11, wherein the subject is a human.

15. The system of claim 11, wherein the target tissue is a body area about 1/100^{th} of the total body mass of the subject.

16. The system of claim 11, wherein the microdose of drug is about 1/100^{th} or less of a total body minimal pharmacological dose for the subject.

17. The system of claim 11, wherein the microdose of drug is about 100 μg or less.

18. The system of claim 11, wherein the drug is one of nitrates, inotropes, analgesics, chemotherapy, anticoagulants, immune modulators, sodium glucose, cotransporter-2 (SGLT-2), inhibitors, central nervous system stimulants, and depressants.

19. The system of claim 11, wherein the sensor is configured to measure a biomarker.

20. The system of claim 19, wherein the biomarker is one of vasodilation, vasoconstriction, analgesia, receptor binding, coagulation parameters, platelet aggregation, cytokines, glucose absorption in proximal tubule, and neuronal activity.
